(19) United States
(12) Patent Application Publication
(10) Pub. No.: US 2024/0215842 A1
(43) Pub. Date: Jul. 4, 2024

(54) BIOMETRIC INFORMATION MEASUREMENT DEVICE AND METHOD OF MANUFACTURING BIOMETRIC INFORMATION MEASUREMENT DEVICE

(71) Applicant: Sharp Semiconductor Innovation Corporation, Tenri City (JP)

(72) Inventors: YUTA MOCHIZUKI, Tenri City (JP); Daisuke Funao, Tenri City (JP); Kohji Kobayashi, Tenri City (JP); Sota Hida, Tenri City (JP); Masayuki Fujio, Tenri City (JP); Keiichi Shibata, Tenri City (JP)

(21) Appl. No.: 18/544,815

(22) Filed: Dec. 19, 2023

(30) Foreign Application Priority Data
Dec. 28, 2022 (JP) 2022-212048

(51) Int. Cl. A61B 5/024

(52) U.S. Cl. CPC A61B 5/02427; A61B 2562/0233; A61B 2562/12

(57) ABSTRACT

A biometric information measurement device includes: a layered film including a plurality of layered layers including two or more types of layers having different refractive indices; and a light receiving element configured to receive light transmitted through the layered film and generate a signal corresponding to the light.

### CROSS-REFERENCE TO RELATED APPLICATIONS

This application claims the benefit of priority to Japanese Patent Application Number 2022-212048 filed on Dec. 28, 2022. The entire contents of the above-identified application are hereby incorporated by reference.

### BACKGROUND

### Technical Field

The present disclosure relates to a biometric information measurement device and a method of manufacturing the biometric information measurement device. The present application claims priority to JP 2022-212048 filed on Dec. 28, 2022, the contents of which are incorporated herein by reference.

WO 2017/104056 discloses a biometric information measurement device.

In the biometric information measurement device, a first pixel receives light in a wavelength range from 530 nm to 590 nm transmitted through a first filter, and generates a first imaging signal. A second pixel receives light in a wavelength range from 500 nm to 530 nm and from 590 nm to 620 nm transmitted through a second filter, and generates a second imaging signal. First time-series data is generated from the first imaging signal, and second time-series data is generated from the second imaging signal. A heartbeat component and an illumination variation component are separated from the first time-series data and the second time-series data. In the wavelength range from 530 nm to 590 nm, hemoglobin has strong absorption properties (paragraphs 0014, 0019 to 0030, and 0037).

### SUMMARY

The spectral transmittance of a filter that transmits light received by a pixel may have an obtuse peak. Therefore, in the biometric information measurement device disclosed in WO 2017/104056, the second time-series data may include the heartbeat component. Thus, both the first time-series data and the second time-series data include the heartbeat component and the illumination variation component. As a result, when the heartbeat component is calculated using the first time-series data and the second time-series data, the heartbeat component and the illumination variation component may not be accurately separated. Thus, biometric information may fail to be accurately measured.

An aspect of the present disclosure has been made in view of the problem described above. It is an object of an aspect of the present disclosure to provide, for example, a biometric information measurement device that can accurately measure biometric information.

A biometric information measurement device according to an aspect of the present disclosure includes a layered film including a plurality of layered layers including two or more types of layers having different refractive indices, and a light receiving element configured to receive light transmitted through the layered film and generate a signal corresponding to the light.

### BRIEF DESCRIPTION OF DRAWINGS

The disclosure will be described with reference to the accompanying drawings, wherein like numbers reference like elements.

FIG. 1 is a block diagram of a biometric information measurement device according to a first embodiment.

FIG. 2 is a cross-sectional view schematically illustrating a module in the biometric information measurement device according to the first embodiment.

FIG. 3 is a plan view schematically illustrating a plurality of pixels in the biometric information measurement device according to the first embodiment.

FIG. 4 is a cross-sectional view schematically illustrating a green pixel and an infrared pixel in the biometric information measurement device according to the first embodiment.

FIG. 5 is a cross-sectional view schematically illustrating a red pixel and a blue pixel in the biometric information measurement device according to the first embodiment.

FIG. 6 is a graph showing spectral molar absorption coefficients of oxyhemoglobin and hemoglobin, as well as eumelanin and pheomelanin which are types of melanin.

FIG. 7 is a cross-sectional view schematically illustrating a layered film in the biometric information measurement device according to the first embodiment.

FIG. 8 is a graph showing an example of spectral transmittance of a layered film provided in a red color filter in the biometric information measurement device according to the first embodiment.

FIG. 9 is a graph showing an example of spectral transmittance of a layered film provided in a green color filter and a blue color filter in the biometric information measurement device according to the first embodiment.

FIG. 10 is a graph showing an example of spectral sensitivity of a red pixel (RE), a green pixel (GR), a blue pixel (BU), and an infrared pixel (IR) in the biometric information measurement device according to the first embodiment.

FIG. 11 is a flowchart illustrating a flow of manufacturing the biometric information measurement device according to the first embodiment.

FIG. 12 is a cross-sectional view schematically illustrating a green pixel and a blue pixel in a biometric information measurement device according to a modification of the first embodiment.

### DESCRIPTION OF EMBODIMENTS

Embodiments of the present disclosure will be described below with reference to the drawings. In the drawings, identical or equivalent elements are given the same reference signs, and redundant descriptions thereof are omitted.

### 1 First Embodiment

### 1.1 Biometric Information Measurement Device

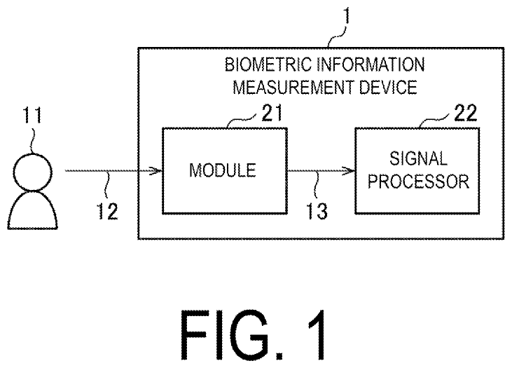

FIG. 1 is a block diagram of a biometric information measurement device according to a first embodiment.

A biometric information measurement device 1 according to the first embodiment illustrated in FIG. 1 receives light 12 from a living body 11 and measures biometric information from the received light 12. The biometric information measurement device 1 measures the biometric information in a non-invasive manner.

The living body 11 is a body of an organism. The organism is, for example, an organism having a circulatory organ that circulates blood containing oxyhemoglobin. The organism having a circulatory organ is a human, for example.

The measured biometric information is information indicating the state of the living body 11. The information includes a pulse volume, a heart rate estimated from the pulse volume, a blood pressure estimated from the pulse volume, and the like. The pulse volume is generated as a result of slight variation in the intensity of light reflected by the skin of the living body 11 due to alternate and repeated expansion and contraction of a blood vessel in which blood flows.

The variation in light intensity caused by the alternate and repeated expansion and contraction of a blood vessel is extremely small. Thus, the amplitude of the pulse volume is small. For this reason, measurement of the pulse volume is affected by noise caused by factors such as body movement, a change in light source, and absorption of light by melanin, which differs greatly between individuals. Therefore, it is generally difficult to measure the pulse wave with high accuracy. The biometric information measurement device 1 overcomes this problem by measuring biometric information such as a pulse wave with high accuracy.

As illustrated in FIG. 1, the biometric information measurement device 1 includes a module 21 and a signal processor 22.

The module 21 receives the light 12 from the living body 11 and generates a signal group 13 corresponding to the received light 12.

The signal processor 22 processes the generated signal group 13 to acquire biometric information.

### 1.2 Module

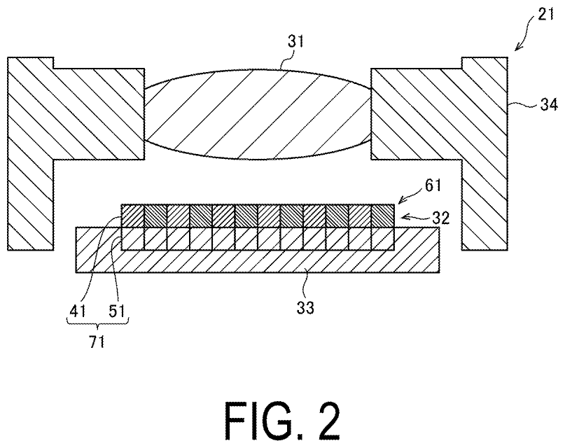

FIG. 2 is a cross-sectional view schematically illustrating a module in the biometric information measurement device according to the first embodiment.

As illustrated in FIG. 2, the module 21 includes a lens 31, a color filter array 32, a solid-state image sensor 33, and a support member 34.

In the module 21, the lens 31, the color filter array 32, the solid-state image sensor 33, and the support member 34 are integrated.

The lens 31 guides the light 12 from the living body 11 to the solid-state image sensor 33. The lens 31 focuses the light 12 on the solid-state image sensor 33 to form an image of the living body 11 on the solid-state image sensor 33. The module 21 may include an optical element other than the lens 31, instead of or in addition to the lens 31. For example, the module 21 may include a prism, a mirror, or a filter. The lens 31 may not be integrated with the color filter array 32 and the solid-state image sensor 33. The biometric information measurement device 1 may have a mechanism enabling the lens 31 to be attached and detached. When the biometric information measurement device 1 has such a mechanism, the user may attach a desired lens to the biometric information measurement device 1.

The color filter array 32 transmits the light guided by the lens 31. The color filter array 32 includes a plurality of color filters 41. The plurality of color filters 41 are two-dimensionally arranged in a plane orthogonal to the optical axis of the lens 31. The plurality of color filters 41 are arranged in a matrix. Each of the plurality of color filters 41 transmits light. Each of the plurality of color filters 41 has wavelength selectivity.

The solid-state image sensor 33 receives the light transmitted through the color filter array 32 and generates the signal group 13 corresponding to the received light. The solid-state image sensor 33 generates the signal group 13 corresponding to the image formed on the solid-state image sensor 33. Therefore, the generated signal group 13 constitutes an image signal of the image. The solid-state image sensor 33 includes a plurality of light receiving elements 51. The plurality of light receiving elements 51 are two-dimensionally arranged in a plane orthogonal to the optical axis of the lens 31. The plurality of light receiving elements 51 are arranged in a matrix. Each of the plurality of light receiving elements 51 receives light and generates a signal corresponding to the received light. The signals generated by the plurality of light receiving elements 51 constitute the signal group 13 generated by the solid-state image sensor 33. The plurality of color filters 41 are disposed on the plurality of light receiving elements 51, respectively. Thus, the plurality of light receiving elements 51 receive a plurality of light beams transmitted through the plurality of color filters 41, respectively, and generate signals corresponding to the plurality of received light beams, respectively. The solid-state image sensor 33 is a complementary metal oxide semiconductor image sensor (CIS). The solid-state image sensor 33 may be an image sensor other than a CIS. For example, the solid-state image sensor 33 may be a charge-coupled device (CCD) image sensor.

As illustrated in FIG. 2, the color filter array 32 and the solid-state image sensor 33 include a pixel array 61. The pixel array 61 includes a plurality of pixels 71. The plurality of pixels 71 include the plurality of light receiving elements 51 and the plurality of color filters 41 disposed on the plurality of light receiving elements 51, respectively.

The support member 34 supports the lens 31.

### 1.3 Pixel

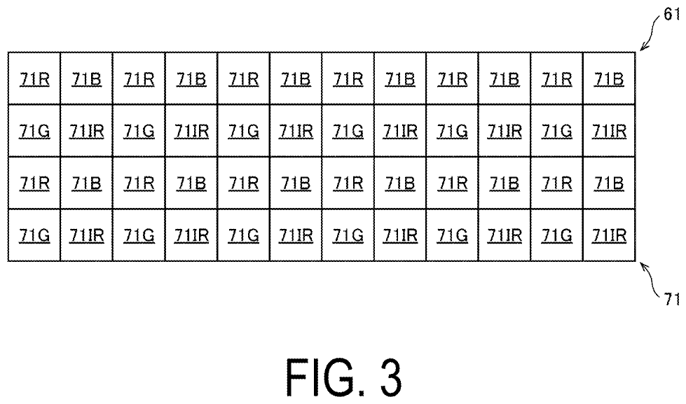

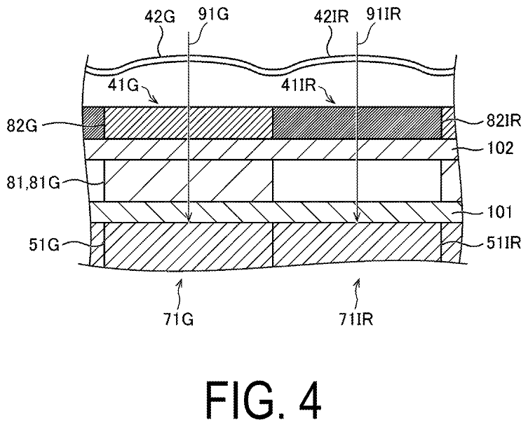

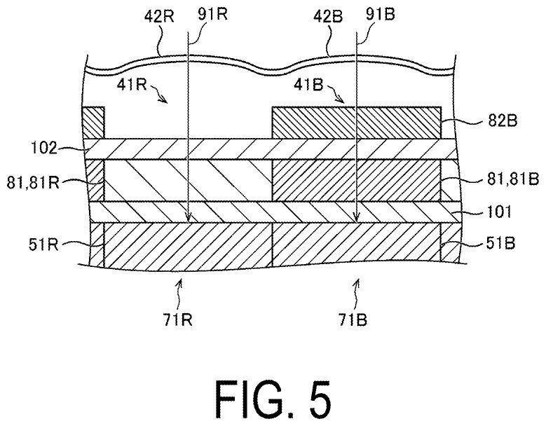

FIG. 3 is a plan view schematically illustrating a plurality of pixels in the biometric information measurement device according to the first embodiment. FIG. 4 is a cross-sectional view schematically illustrating a green pixel and an infrared pixel in the biometric information measurement device according to the first embodiment. FIG. 5 is a cross-sectional view schematically illustrating a red pixel and a blue pixel in the biometric information measurement device according to the first embodiment.

As illustrated in FIGS. 3, 4, and 5, the plurality of pixels 71 include red pixels 71R, green pixels 71G, blue pixels 71B, and infrared pixels 71IR. The plurality of light receiving elements 51 include light receiving elements 51R, 51G, 51B, and 51IR. The plurality of color filters 41 include a red color filter 41R, a green color filter 41G, a blue color filter 41B, and an infrared color filter 41IR.

Each of the red pixel 71R, the green pixel 71G, and the blue pixel 71B has a spectral sensitivity that is at a maximum in a visible light wavelength band from 370 nm to 740 nm. The infrared pixel 71IR has a spectral sensitivity that is at a maximum in the infrared wavelength band.

The red pixels 71R, the green pixels 71G, the blue pixels 71B, and the infrared pixels 71IR are periodically arrayed. The red color filters 41R, the green color filters 41G, the blue color filters 41B, and the infrared color filters 41IR are periodically arrayed.

The red pixel 71R, the green pixel 71G, the blue pixel 71B, and the infrared pixel 71IR include the light receiving elements 51R, 51G, 51B, and 51IR, respectively, and include the red color filter 41R, the green color filter 41G, the blue color filter 41B, and the infrared color filter 41IR, respectively. The red color filter 41R, the green color filter 41G, the blue color filter 41B, and the infrared color filter 41IR are disposed on the light receiving elements 51R, 51G, 51B, and 51IR, respectively. Thus, the red color filter 41R, the green color filter 41G, the blue color filter 41B, and the infrared color filter 41IR are disposed on optical paths of light beams 91R, 91G, 91B, and 91IR to be received by the light receiving elements 51R, 51G, 51B, and 51IR, respectively. The red color filter 41R, the green color filter 41G, the blue color filter 41B, and the infrared color filter 41IR have higher wavelength selectivity than that of an absorption-type filter.

The red color filter 41R, the green color filter 41G, the blue color filter 41B, and the infrared color filter 41IR have different spectral transmittances. Therefore, the plurality of color filters 41 include four types of color filters having different spectral transmittances.

The red pixel 71R, the green pixel 71G, the blue pixel 71B, and the infrared pixel 71IR have spectral sensitivities corresponding to the spectral transmittances of the red color filter 41R, the green color filter 41G, the blue color filter 41B, and the infrared color filter 41IR, respectively. Therefore, the red pixel 71R, the green pixel 71G, the blue pixel 71B, and the infrared pixel 71IR have different spectral sensitivities. For this reason, the plurality of pixels 71 include four types of pixels having different spectral sensitivities.

The number of types of color filters having different spectral transmittances may be three or less or five or more. Therefore, the number of types of pixels having different spectral sensitivities may be three or less or five or more. All of the plurality of color filters 41 may have the same spectral transmittance. Therefore, all of the plurality of pixels 71 may have the same spectral sensitivity.

The red color filter 41R selectively transmits red (R) light. The green color filter 41G selectively transmits green (G) light. The blue color filter 41B selectively transmits blue (B) light. The infrared color filter 41IR selectively transmits infrared (IR) light. Therefore, the red pixel 71R has high sensitivity to red light. The green pixel 71G has high sensitivity to green light. The blue pixel 71B has high sensitivity to blue light. The infrared pixel 71IR has high sensitivity to infrared light.

As described above, the number of types of pixels having different spectral sensitivities in the plurality of pixels 71 may be three or less. Therefore, the pixels having different spectral sensitivities in the plurality of pixels 71 may be only the red pixel 71R and the infrared pixel 71IR, or may be only the red pixel 71R and the green pixel 71G.

### 1.4 Improvement of Wavelength Selectivity of Color Filter

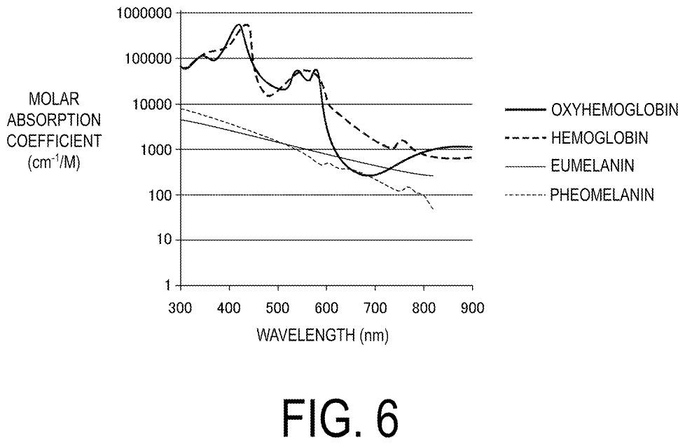

FIG. 6 is a graph showing spectral molar absorption coefficients of oxyhemoglobin and hemoglobin, as well as eumelanin and pheomelanin which are types of melanin. In the graph, the horizontal axis represents the wavelength and the vertical axis represents the molar absorption coefficient.

The pulse volume is mainly a result of a temporal change in light absorption by oxyhemoglobin contained in blood flowing through a blood vessel. As illustrated in FIG. 6, the absorption coefficient of oxyhemoglobin is large in the wavelength band from 400 nm and in the wavelength band from 530 nm to 590 nm, and is small in the wavelength band from 620 nm to 740 nm. For this reason, to measure the pulse volume with high accuracy, it is desirable to acquire the pulse volume from light having a wavelength in the wavelength band around 400 nm or in the wavelength band from 530 nm to 590 nm where the absorption coefficient of oxyhemoglobin is large. However, as illustrated in FIG. 6, since the absorption coefficient of melanin is also large in the wavelength band around 400 nm, to measure the pulse volume with high accuracy, it is more desirable to acquire the pulse volume from light having a wavelength in the wavelength band from 530 nm to 590 nm where the absorption coefficient of melanin is small.

In a solid-state image sensor including a red pixel, a green pixel, and a blue pixel having spectral sensitivity optimized for human visual sensitivity characteristics, a pixel having high sensitivity in a wavelength band in which the absorption coefficient of oxyhemoglobin is large is the green pixel. For this reason, when the pulse volume is acquired from the signal group generated by the solid-state image sensor, the pulse volume is acquired from the signal generated by the green pixel. However, when the spectral sensitivity of the green pixel has an obtuse peak, the green pixel has high sensitivity also in a wavelength band in which the absorption coefficient of oxyhemoglobin is small. For this reason, when the spectral sensitivity of the green pixel has an obtuse peak, the pulse volume cannot be measured with high accuracy.

In view of this problem, the biometric information measurement device 1 has the wavelength selectivity of the green color filter 41G and the blue color filter 41B improved such that the green pixel 71G and the blue pixel 71B have high sensitivity in a wavelength band in which the absorption coefficient of oxyhemoglobin is large and do not have high sensitivity in other wavelength bands. Thus, the biometric information measurement device 1 measures pulse volume with high accuracy.

As described above, the amplitude of the pulse volume is small. For this reason, the measurement of the pulse volume is affected by noise caused by factors such as body movement, a change in light source, and absorption of light by melanin, which differs greatly between individuals. Thus, to measure the pulse volume with high accuracy, it is desirable to remove noise from the pulse volume by using light having a wavelength in a wavelength band in which the absorption coefficient of oxyhemoglobin is small.

In a solid-state image sensor including a red pixel, a green pixel, and a blue pixel having spectral sensitivity optimized for human visual sensitivity characteristics, a pixel having high sensitivity in a wavelength band in which the absorption coefficient of oxyhemoglobin is small is a pixel other than the green pixel, and is the red pixel in particular. For this reason, when the pulse volume is acquired from the signal group generated by the solid-state image sensor, noise is removed from the pulse volume using a signal generated by a pixel other than the green pixel. However, when the spectral sensitivity of the pixel other than the green pixel has an obtuse peak, the pixel other than the green pixel has high sensitivity also in a wavelength band in which the absorption coefficient of oxyhemoglobin is large. Thus, when the spectral sensitivity of the pixel other than the green pixel has an obtuse peak, the pulse volume is offset when the noise is removed from the pulse volume. For this reason, when the spectral sensitivity of the pixel other than the green pixel has an obtuse peak, the pulse volume cannot be measured with high accuracy.

In view of this problem, the biometric information measurement device 1 has the wavelength selectivity of the red color filter 41R improved such that the red pixel 71R has high sensitivity in a wavelength band in which the absorption coefficient of oxyhemoglobin is small and does not have high sensitivity in other wavelength bands. Thus, the biometric information measurement device 1 appropriately removes noise from the pulse volume.

### 1.5 Structure of Color Filter

As illustrated in FIG. 5, the red color filter 41R includes a layered film 81R.

The layered film 81R is an interference-type filter having higher wavelength selectivity than that of an absorption-type filter. Accordingly, the layered film 81R has high wavelength selectivity.

The layered film 81R is disposed on the light receiving element 51R. Accordingly, the layered film 81R is disposed on the optical path of the light beam 91R received by the light receiving element 51R. Thus, the light receiving element 51R receives the light beam 91R transmitted through the layered film 81R and generates a signal corresponding to the received light beam 91R.

As illustrated in FIG. 4, the green color filter 41G includes an absorption-type filter 82G and a layered film 81G.

The absorption-type filter 82G absorbs light other than green light and selectively transmits green light.

The layered film 81G is an interference-type filter. Thus, the layered film 81G absorbs or reflects light other than light having a specific wavelength and selectively transmits the light having the specific wavelength. The layered film 81G has higher wavelength selectivity than that of the absorption-type filter 82G. Thus, the layered film 81G transmits light having a range of wavelengths narrower than the range of wavelengths of the light transmitted through the absorption-type filter 82G.

The layered film 81G is disposed on the light receiving element 51G. The absorption-type filter 82G is disposed on the layered film 81G. Accordingly, the layered film 81G and the absorption-type filter 82G are disposed on the optical path of the light beam 91G received by the light receiving element 51G. Thus, the light receiving element 51G receives the light beam 91G transmitted through the absorption-type filter 82G and the layered film 81G and generates a signal corresponding to the received light beam 91G.

As illustrated in FIG. 5, the blue color filter 41B includes an absorption-type filter 82B and a layered film 81B.

The absorption-type filter 82B absorbs light other than blue light and selectively transmits blue light.

The layered film 81B is an interference-type filter. Thus, the layered film 81B absorbs or reflects light other than light having a specific wavelength, and selectively transmits the light having the specific wavelength. The layered film 81B has higher wavelength selectivity than that of the absorption-type filter 82B. Thus, the layered film 81B transmits light having a range of wavelengths narrower than the range of wavelengths of the light transmitted through the absorption-type filter 82B.

The layered film 81B is disposed on the light receiving element 51B. The absorption-type filter 82B is disposed on the layered film 81B. Accordingly, the layered film 81B and the absorption-type filter 82B are disposed on the optical path of the light beam 91B received by the light receiving element 51B. Thus, the light receiving element 51B receives the light beam 91B transmitted through the absorption-type filter 82B and the layered film 81B and generates a signal corresponding to the received light beam 91B.

As illustrated in FIG. 4, the infrared color filter 41IR includes an absorption-type filter 82IR.

The absorption-type filter 82IR is disposed on the light receiving element 51IR. Accordingly, the absorption-type filter 82IR is disposed on the optical path of the light beam 91IR received by the light receiving element 51IR. Thus, the light receiving element 51IR receives the light beam 91IR transmitted through the absorption-type filter 82IR and generates a signal corresponding to the received light beam 91IR.

The absorption-type filter 82IR absorbs light other than infrared light and selectively transmits infrared light.

The layered films 81R, 81G, and 81B are made of an inorganic material. The absorption-type filters 82G, 82B, and 82IR are made of at least one type selected from the group consisting of an organic material and an inorganic material.

The color filter array 32 includes planarization films 101 and 102. The module 21 includes microlenses 42R, 42G, 42B and 42IR.

The planarization film 101 is disposed on the plurality of pixels 71. The planarization film 101 spreads entirely over the plurality of pixels 71. The planarization film 101 has a flat upper surface. The layered films 81R, 81G, and 81B are disposed on the upper surface.

The planarization film 102 is disposed on the plurality of pixels 71, overlapping the planarization film 101 and the layered films 81R, 81G, and 81B. The planarization film 102 spreads entirely over the plurality of pixels 71. The planarization film 102 has a flat upper surface. The absorption-type filters 82G, 82B, and 82IR are disposed on the upper surface. The planarization film 102 is disposed between the layered films 81R, 81G, and 81B and the absorption-type filters 82G, 82B, and 82IR. Thus, the planarization film 102 serves as a spacer member that separates the layered films 81R, 81G, and 81B and the absorption-type filters 82G, 82B, and 82IR from each other.

The microlenses 42R, 42G, 42B and 42IR are disposed on the red color filter 41R, the green color filter 41G, the blue color filter 41B, and the infrared color filter 41IR, respectively.

The light beams 91R, 91G, 91B, and 91IR pass through the microlenses 42R, 42G, 42B, and 42IR, respectively, and pass through the planarization films 101 and 102.

### 1.6 Structure of Layered Film

As illustrated in FIG. 4 and FIG. 5, the biometric information measurement device 1 includes the plurality of layered films 81R, 81G, and 81B, and the plurality of light receiving elements 51R, 51G, and 51B. The plurality of light receiving elements 51R, 51G, and 51B receive the light beams 91R, 91G, and 91B that have passed through the plurality of layered films 81R, 81G, and 81B, respectively, and generate signals corresponding to the light beams 91R, 91G, and 91B, respectively. The plurality of layered films 81R, 81G, and 81B have different wavelength selectivities.

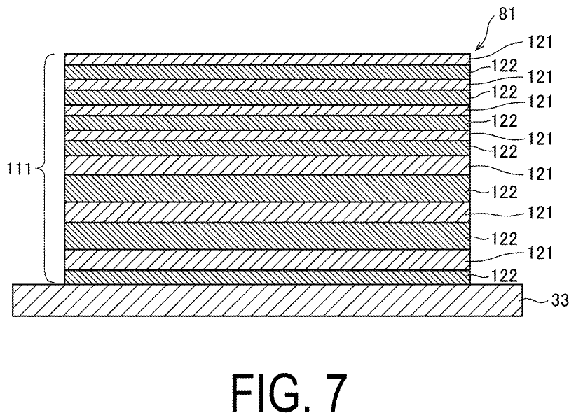

FIG. 7 is a cross-sectional view schematically illustrating a layered film in the biometric information measurement device according to the first embodiment.

As illustrated in FIG. 7, each layered film 81, that is, each of the layered films 81R, 81G, and 81B includes a plurality of layers 111.

The plurality of layers 111 include two types of layers 121 and 122 having different refractive indices. The plurality of layers 111 are layered. With this configuration, the light passing through each layered film 81 is reflected at the interface between the two types of layers 121 and 122, and the reflected light beams interfere with each other. Thus, each of the layered films 81 transmits light in one specific wavelength band and hardly transmits light in another specific wavelength band. Thus, each layered film 81 has high wavelength selectivity. The plurality of layers 111 may include three or more types of layers having different refractive indices.

Each of the plurality of layers 111 has a thickness equal to or greater than 20 nm and equal to or less than 500 nm. The plurality of layers 111 is preferably five or more layers. With such configurations, it is easy to obtain the layered film 81 having high wavelength selectivity. While the upper limit of the number of the plurality of layers 111 is not limited, the plurality of layers 111 is, for example, 30 layers or less.

The wavelength selectivity of each layered film 81 depends on a combination of the thickness and the number of the plurality of layers 111 in each layered film 81. The plurality of layered films 81R, 81G, and 81B have different wavelength selectivities. Thus, the combination of the thickness and the number of the plurality of layers 111 is different between the plurality of layered films 81R, 81G, and 81B.

### 1.7 Spectral Transmittance of Layered Film Provided in Red Color Filter

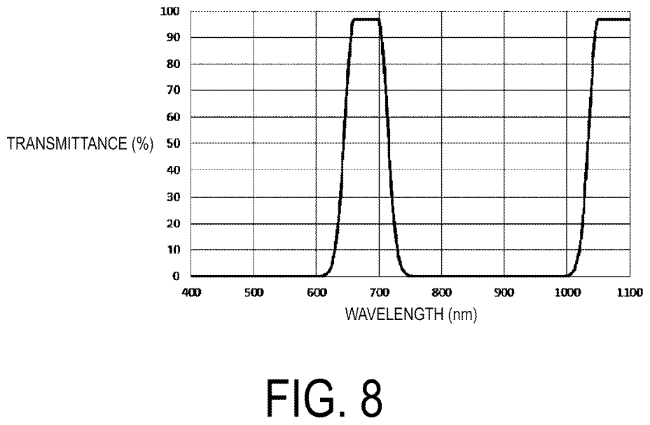

FIG. 8 is a graph showing an example of spectral transmittance of a layered film provided in a red color filter in the biometric information measurement device according to the first embodiment. In the graph, the horizontal axis represents the wavelength and the vertical axis represents the transmittance.

As shown in FIG. 8, the layered film 81R has a spectral transmittance that is at a maximum at a wavelength equal to or longer than 620 nm and equal to or shorter than 740 nm. Accordingly, the red pixel 71R including the layered film 81R can have high sensitivity in the wavelength band from 620 nm to 740 nm.

The absorption coefficient of oxyhemoglobin is small in the wavelength band from 620 nm to 740 nm. Accordingly, a pulse volume can hardly be acquired from light having a wavelength in that wavelength band. Thus, the signal generated by the light receiving element 51R that receives the light beam 91R transmitted through the layered film 81R is suitably used as a reference signal for removing noise from the pulse volume. For example, the signal generated by the light receiving element 51R is suitably used as the reference signal for removing noise from the pulse volume acquired from the signal generated by the light receiving element 51G that receives the light beam 91G transmitted through the layered film 81G or the light receiving element 51B that receives the light beam 91B transmitted through the layered film 81B. With this configuration, it is possible to suppress the impact of noise caused by factors such as body movement, a change in light source, and absorption of light by melanin, which differs greatly between individuals.

The long-wavelength-side spectral transmittance of the layered film 81R is designed based on the absorption coefficient of a photodiode in the light receiving element 51R. For example, when the quantum efficiency of the photodiode is sufficiently low at a wavelength that is equal to or longer than 1000 nm, the long-wavelength-side spectral transmittance of the layered film 81R is designed to be low at a wavelength shorter than 1000 nm and high at a wavelength equal to or longer than 1000 nm, as shown in FIG. 8. Still, the wavelength at which the absorption coefficient of the photodiode is small changes depending on the conditions on the solid-state image sensor 33 side. Therefore, the wavelengths at which the long-wavelength-side spectral transmittance of the layered film 81R is high are not particularly limited.

### 1.8 Spectral Transmittance of Layered Film Provided in Green Color Filter and Blue Color Filter

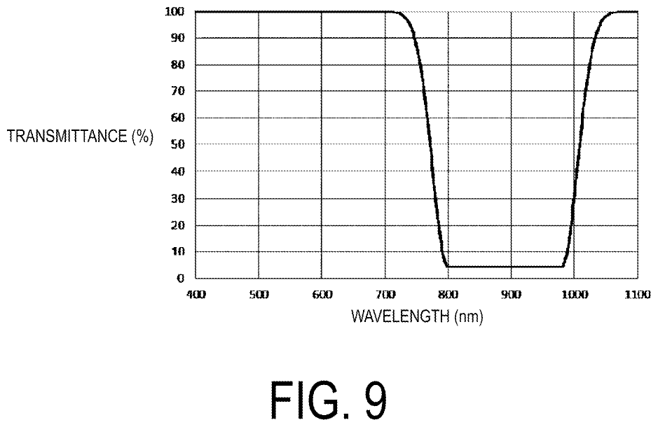

FIG. 9 is a graph showing an example of spectral transmittance of a layered film provided in a green color filter and a blue color filter in the biometric information measurement device according to the first embodiment. In the graph, the horizontal axis represents the wavelength and the vertical axis represents the transmittance.

As shown in FIG. 9, the layered films 81G and 81B have spectral transmittances that are at a minimum at a wavelength equal to or longer than 760 nm and equal to or shorter than 1100 nm. Thus, it is possible to reduce the sensitivity of the green pixel 71G and the blue pixel 71B respectively including the layered films 81G and 81B in the wavelength band from 760 nm to 1100 nm.

The absorption coefficient of oxyhemoglobin is large in the visible light wavelength range and is small in a near-infrared wavelength range. Thus, to acquire a pulse volume having a large amplitude, it is desirable to acquire a pulse volume from light from which a wavelength component having a wavelength in the near-infrared wavelength range is removed. Accordingly, the signals generated by the light receiving elements 51G and 51B that receive the light transmitted through the layered films 81G and 81B, respectively, are suitably used as signals for acquiring pulse volume.

The absorption-type filters 82G and 82B have relatively high transmittance in the near-infrared wavelength range. However, in the green pixel 71G and the blue pixel 71B, the absorption-type filters 82G and 82B having a relatively high transmittance in the near-infrared wavelength range respectively overlap the layered films 81G and 81B having only a low transmittance in the near-infrared wavelength range. Accordingly, the green pixel 71G and the blue pixel 71B having only low sensitivity in the near-infrared wavelength range can be obtained.

The long-wavelength-side spectral transmittance of the layered films 81G and 81B is designed based on the absorption coefficient of the photodiode in the light receiving elements 51G and 51B. For example, when the quantum efficiency of the photodiode is sufficiently low at a wavelength equal to or longer than 1000 nm, the long-wavelength-side spectral transmittance of the layered films 81G and 81B is designed to be low at a wavelength shorter than 1000 nm and high at a wavelength equal to or longer than 1000 nm, as shown in FIG. 9. Still, the wavelength at which the absorption coefficient of the photodiode is small changes depending on the conditions on the solid-state image sensor 33 side. Therefore, the wavelengths at which the long-wavelength-side spectral transmittance of the layered films 81G and 81B is high are not particularly limited.

### 1.9 Spectral Sensitivity of Pixel

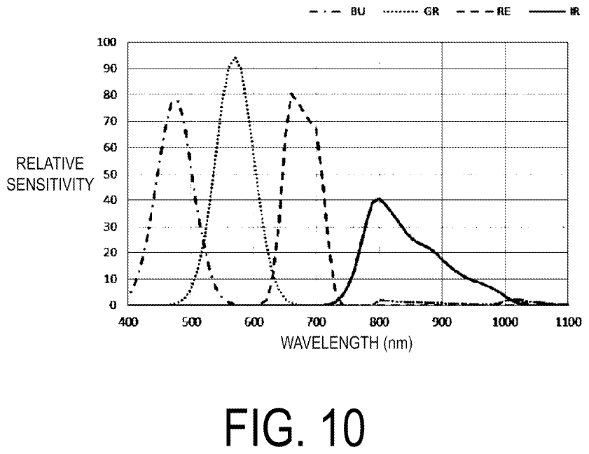

FIG. 10 is a graph showing an example of spectral sensitivity of a red pixel (RE), a green pixel (GR), a blue pixel (BU), and an infrared pixel (IR) in the biometric information measurement device according to the first embodiment.

As shown in FIG. 10, the red pixel 71R has high sensitivity only in the wavelength band from 620 nm to 740 nm. Thus, the signal generated by the red pixel 71R is suitably used as a reference signal for removing noise from the pulse volume.

As shown in FIG. 10, the green pixel 71G and the blue pixel 71B have high sensitivity in the visible light region where the absorption coefficient of oxyhemoglobin is large, and have only low sensitivity in the wavelength band from 760 nm to 1100 nm where the absorption coefficient of oxyhemoglobin is small. Accordingly, the signals generated by the green pixel 71G and the blue pixel 71B are suitably used as signals for acquiring a pulse volume having a large amplitude.

With such configurations, the color filter array 32 and the solid-state image sensor 33 constitute a solid-state image sensor suitably used for measurement of biometric information such as a pulse volume. Incorporating the solid-state image sensor into the biometric information measurement device 1 makes it possible to generate a signal suitable for acquiring a pulse volume having a large amplitude, and to generate a reference signal suitable for removing noise caused by factors such as body movement, a change in light source, and absorption of light by melanin, which differs greatly between individuals. As a result, the pulse volume can be accurately measured, and heart rate, blood pressure, and the like can be accurately estimated from the pulse volume.

### 1.10 Method of Manufacturing Biometric Information Measurement Device

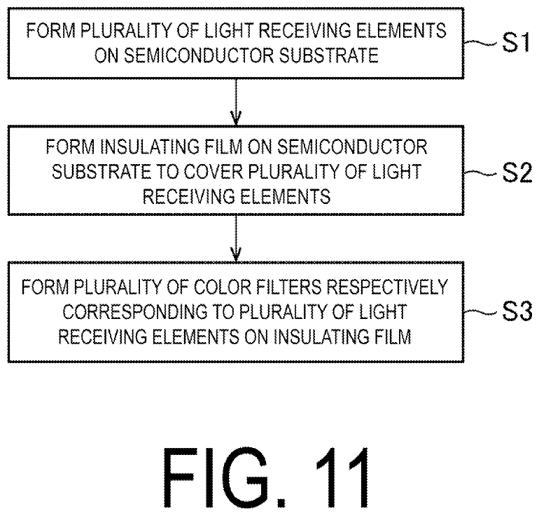

FIG. 11 is a flowchart illustrating a flow of manufacturing the biometric information measurement device according to the first embodiment.

Steps S1 to S3 illustrated in FIG. 11 are executed for manufacturing the biometric information measurement device according to the first embodiment.

In step S1, the plurality of light receiving elements 51 are formed on a semiconductor substrate. Thus, the solid-state image sensor 33 is manufactured.

In the subsequent step S2, an insulating film is formed on the semiconductor substrate to cover the plurality of light receiving elements 51.

In the subsequent step S3, the plurality of color filters 41 respectively corresponding to the plurality of light receiving elements 51 are formed on the insulating film. The plurality of color filters 41 to be formed include an optical filter having a spectral transmittance that is at a maximum at a wavelength at least equal to or longer than 620 nm and equal to or shorter than 740 nm or a spectral transmittance that is at a minimum at a wavelength at least equal to or longer than least 760 nm and equal to or shorter than 1100 nm. The optical filter having a spectral transmittance that is at a maximum at a wavelength at least equal to or longer than 620 nm and equal to or shorter than 740 nm is the layered film 81R. The optical filter having a spectral transmittance that is at a minimum at a wavelength at least equal to or longer than 760 nm and equal to or shorter than 1100 nm is the layered films 81G and 81B.

### 1.10 Modification

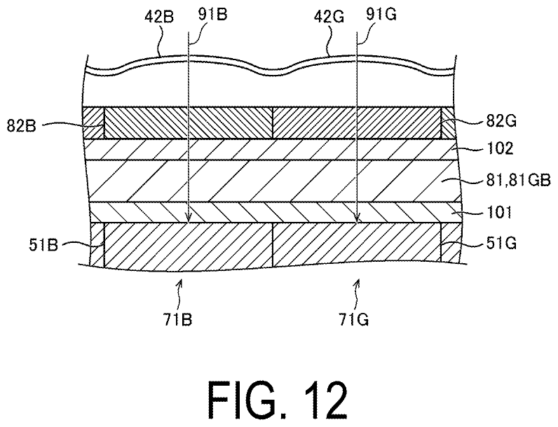

FIG. 12 is a cross-sectional view schematically illustrating a green pixel and a blue pixel in the biometric information measurement device according to a modification of the first embodiment.

In the modification, the light receiving elements 51G and 51B are respectively first and second light receiving elements adjacent to each other, the light beam 91G and the light beam 91B are respectively first and second light beams, the absorption-type filters 82G and 82B are respectively first and second absorption-type filters adjacent to each other, and the signals generated by the light receiving elements 51G and 51B are respectively first and second signals.

The light receiving elements 51G and 51B are adjacent to each other. Thus, the green pixel 71G and the blue pixel 71B are also adjacent to each other.

The color filter array 32 includes a layered film 81GB common to the light receiving elements 51G and 51B.

The light receiving element 51G receives the light beam 91G transmitted through the absorption-type filter 82G and the common layered film 81GB and generates a signal corresponding to the received light beam 91G. The light receiving element 51B receives the light beam 91B transmitted through the absorption-type filter 82B and the common layered film 81GB and generates a signal corresponding to the received light beam 91B.

With such configurations, the effective region of the common layered film 81GB can be expanded to the vicinity of the boundary between the green pixel 71G and the blue pixel 71B.

### 1.11 Measurement Procedure for Biometric Information by Biometric Information Measurement Device

When the biometric information measurement device 1 measures the biometric information, the module 21 receives the light 12 from the living body 11 and generates the signal group 13 corresponding to the image of the living body 11 formed by the received light. Thus, the module 21 captures an image of the living body 11. The module 21 captures the image of the living body 11 in such a manner that the captured image of the living body 11 includes a region including the skin of the living body 11. For example, the module 21 captures an image of the living body 11 including a region including the face, a hand, or the like.

Subsequently, the signal processor 22 executes signal processing on the first signal, the second signal, a third signal, and a fourth signal respectively generated by the red pixel 71R, the green pixel 71G, the blue pixel 71B, and the infrared pixel 71IR used for capturing the image of the region. The signal processing executed includes signal processing for averaging each of the generated first signal, second signal, third signal, and fourth signal. The signal processing executed may include signal processing other than the signal processing for averaging each of the generated first signal, second signal, third signal, and fourth signal. For example, the signal processing executed may include processing for acquiring a median value of each of the generated first signal, second signal, third signal, and fourth signal.

The signal processor 22 removes noise from the pulse volume by performing numerical processing on the first signal, the second signal, the third signal, and the fourth signal on which the signal processing has been executed. The noise to be removed is caused by factors such as body movement, a change in light source, and absorption of light by melanin, which differs greatly between individuals. The signal processing executed includes multiplying the first signal, the second signal, the third signal, and the fourth signal after the signal processing by a coefficient, and performing a calculation as a combination of addition and subtraction on the signals obtained by the multiplication by the coefficient. During the numerical processing, processing for obtaining the logarithm of the signal may be performed to facilitate the calculation.

The signal processor 22 arranges the signals obtained by executing the numerical processing in a time series. Thus, the signal processor 22 can acquire the waveform of the pulse volume.

Note that the present disclosure is not limited to the above embodiments and can be replaced by a configuration that is substantially the same as that described in the above embodiments, a configuration that achieves substantially the same operation and effect, or a configuration that can achieve the same object.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

1. A biometric information measurement device comprising:
a layered film including a plurality of layered layers including two or more types of layers having different refractive indices; and
a light receiving element configured to receive light transmitted through the layered film and generate a signal corresponding to the light.

2. The biometric information measurement device according to claim 1,
wherein each of the plurality of layers has a thickness equal to or greater than 20 nm and equal to or less than 500 nm.

3. The biometric information measurement device according to claim 1,
wherein the layered film has a spectral transmittance that is at a maximum at a wavelength equal to or longer than 620 nm and equal to or shorter than 740 nm.

4. The biometric information measurement device according to claim 1,
wherein the layered film has a spectral transmittance that is at a minimum at a wavelength equal to or longer than 760 nm and equal to or shorter than 1100 nm.

5. The biometric information measurement device according to claim 1, further comprising:
an absorption-type filter made of at least one type selected from the group consisting of an organic material and an inorganic material,
wherein the light passes through the absorption-type filter.

6. The biometric information measurement device according to claim 5,
wherein the light receiving element is a first light receiving element, the light is first light, the signal is a first signal, and the absorption-type filter is a first absorption-type filter, and
the biometric information measurement device further comprises:
a second absorption-type filter made of at least one type selected from the group consisting of an organic material and an inorganic material; and
a second light receiving element configured to receive second light transmitted through the second absorption-type filter and the layered film and generate a second signal based on the second light, the second light receiving element being adjacent to the first light receiving element.

7. The biometric information measurement device according to claim 5, further comprising
a spacer member disposed between the layered film and the absorption-type filter.

8. A method of manufacturing a biometric information measurement device, the method comprising:
forming a light receiving element on a semiconductor substrate;
forming an insulating film on the semiconductor substrate to cover the light receiving element; and
forming an optical filter corresponding to the light receiving element on the insulating film, wherein
the optical filter has a spectral transmittance that is at a maximum at a wavelength at least equal to or longer than 620 nm and equal to or shorter than 740 nm, and a spectral transmittance that is at a minimum at a wavelength at least equal to or longer than 760 nm and equal to or shorter than 1100 nm.
